(19) United States
(12) Patent Application Publication
(10) Pub. No.: US 2015/0050348 A1
(43) Pub. Date: Feb. 19, 2015

(54) BIOMECHANICAL FORCE MITIGATION IN THE DELIVERY OF STEM CELL THERAPIES

(71) Applicants: Raymond P. Vito, Atlanta, GA (US); Jack C. Griffis, Decatur, GA (US); W. Robert Taylor, Stone Mountain, GA (US); Michael L. Wach, Alpharetta, GA (US)

(72) Inventors: Raymond P. Vito, Atlanta, GA (US); Jack C. Griffis, Decatur, GA (US); W. Robert Taylor, Stone Mountain, GA (US); Michael L. Wach, Alpharetta, GA (US)

(21) Appl. No.: 13/969,317

(22) Filed: Aug. 16, 2013

(51) Int. Cl. A61K 35/12; A61K 9/00

(52) U.S. Cl. CPC A61K 35/12; A61K 9/00
USPC 424/490; 424/93.7

(57) ABSTRACT

A medical practitioner can specify certain parameters for a procedure that involves delivering a therapeutic agent, while leaving other parameters open. The therapeutic agent can be sensitive to biomechanical forces (or other influences) associated with delivery. The procedure can involve regenerative medicine, for example delivering progenitor or stem cells to a diseased heart using a catheter, whereby unbridled transport in the catheter may compromise efficacy. The open parameters can influence efficacy of the agent and thus therapeutic outcome. A computer-based system can apply stored information, such as from databases, to narrow the possible values of the open parameters. From the narrowed possibilities, an optimization routine can determine suitable or optimized values for the open parameters. The determined values can manage biomechanical forces incurred by the therapeutic agent, thereby promoting efficacy and healing. The optimized parameters can guide the practitioner in the procedure.

### RELATED APPLICATION

This application is a continuation of and claims priority to U.S. patent application Ser. No. 13/466,041, filed May 7, 2012 and entitled “Guided Delivery of Cell, Particle, and Drug Based Therapies,” which was a continuation of and claimed priority to U.S. patent application Ser. No. 12/454,396, filed May 18, 2009 and entitled “Method and System for Mitigating Effects of Biomechanical Forces on Cell, Particle and Drug Based Therapies.” The complete disclosure of the two above-identified priority applications are hereby fully incorporated herein by reference.

### FIELD OF THE TECHNOLOGY

The present technology is generally related to treating disease or other malady using cells, drugs, particles or combinations thereof as therapeutic agents, and more specifically to managing the delivery of such therapeutic agents to avoid compromising efficacy due to biomechanical forces incurred during delivery.

### BACKGROUND

The processes used by interventionalists when employing traditional therapeutic agents in the treatment of human disease or medical malady are substantially well established and understood. Typically, an interventionalist chooses a therapeutic agent, a dosage and a delivery means or delivery device or system appropriate for treatment of a particular human disease or medical malady. An example is routinely selecting an orally delivered antibiotic, its dosage and the periodicity of delivery for treating a known bacterial infection. However, such status quo procedures are often ill suited to a new class of therapeutic agents that are very sensitive to delivery parameters.

Cells, large molecule drugs and particles or combinations thereof are emerging as therapeutic agents with tremendous potential. Studies indicate that such agents, unlike most of their traditional counterparts, can be affected in complex ways by biomechanical forces and environmental factors associated with their handling and, more significantly, with their delivery to an organ, organs or organ system(s) in connection with a treatment regime. Although handling, environment, and biomechanical forces can dramatically influence efficacy of many new agents, the art lacks a systematic way to inform decision making associated with their prescription, administration and delivery. Moreover, the art lacks adequate systems and methods for planning, guiding or otherwise helping a person (or an inanimate system) manipulate, transport, handle, and/or move a therapeutic agent whose therapeutic effectiveness may be sensitive to such actions. A technology addressing this need, or some other related technological deficit, would benefit medicine.

### SUMMARY

In one aspect of the present invention, a computer-based system or process can preferentially select or optimize parameters or conditions associated with delivering a therapeutic agent to a patient to avoid unintentionally changing a therapeutic effect of the agent as a result of biomechanical forces applied to the agent during delivery. For example, a computer-based system or process can advise or guide an interventionalist, or some other practitioner or user, about how to deliver a sensitive therapeutic agent to achieve healing.

The discussion of delivering therapeutic agents presented in this summary is for illustrative purposes only. Various aspects of the present invention may be more clearly understood and appreciated from a review of the following detailed description of the disclosed embodiments and by reference to the figures and claims. Other aspects, systems, processes, methods, features, advantages, benefits, and objects of the present invention will become apparent to one of ordinary skill in the art upon examination of the following detailed description and the accompanying figures. It is intended that all such aspects, systems, processes, methods, features, advantages, benefits, and objects are to be included within this description, are to be within the scope of the present invention, and are to be protected by the accompanying claims.

### BRIEF DESCRIPTION OF THE FIGURES

FIG. 1 illustrates a plot for a representative therapeutic agent of data that can inform a process or system for mitigating biomechanical force dependent effects on a therapeutic agent in accordance with certain exemplary embodiments of the present invention. The represented data might be obtained from experiments, models or combinations thereof, for example.

FIG. 2 illustrates an interventionalist treating a patient and using a system and process to guide or control delivery of a therapeutic agent to mitigate biomechanical force dependent effects in accordance with certain exemplary embodiments of the present invention.

FIG. 3 illustrates a block diagram of data flow and logic for a process used to inform decision making for delivery of cell, drug and particle based therapies while maintaining their efficacy in accordance with certain exemplary embodiments of the present invention.

FIG. 4 illustrates a schematic for a representative embodiment of a process for mitigating biomechanical force dependent effects on a therapeutic agent in accordance with certain exemplary embodiments of the present invention.

FIG. 5 illustrates a flowchart for a routine component executed as part of a process for mitigating biomechanical force dependent effects on a therapeutic agent in accordance with certain exemplary embodiments of the present invention

FIG. 6 illustrates a diagram of representative logic for a routine component of FIG. 5 in accordance with certain exemplary embodiments of the present invention.

Many aspects of the present invention can be better understood with reference to the above figures. The elements and features shown in the figures are not necessarily to scale, emphasis instead being placed upon clearly illustrating principles of exemplary embodiments of the present invention. Moreover, certain dimensions may be exaggerated to help visually convey such principles. In the figures, reference numerals designate like or corresponding, but not necessarily identical, elements throughout the several views.

### DETAILED DESCRIPTION OF EXEMPLARY EMBODIMENTS

Certain exemplary embodiments of the present invention can facilitate treating disease or other malady using delivery sensitive therapeutic agents, such as cells, drugs, particles or combinations thereof. These agents are typically delivered directly to a specific organ, organs or organ system(s) in need of treatment. Examples of organs in need of treatment include the human heart and/or blood vessels. Atherosclerosis is one example of a disease that is treatable with such agents, with stem cells, progenitor cells and drugs encapsulated in nanoparticles becoming recognized as useful therapeutic agents for the disease. Delivery of the therapeutic agent may, for example, comprise injection through a needle, a catheter, or a combination of catheter(s) and needle(s). Biomechanical forces, such as shear stress, often inadvertently and unavoidably associated with delivery of these therapeutic agents, can affect their efficacy as treatments. Efficacy may also be affected by environmental conditions associated with handling, for example temperature and processing time or means. As discussed in further detail below, certain exemplary embodiments of the present invention promote successful treatment in such an application through planning and/or guiding treatment decisions and actions.

Cells or drugs—particularly those having high molecular weight—or combinations of drugs and cells are emerging as potent and important therapeutic agents for the treatment of human disease. Particles of varying composition, size (e.g., nano particles or micro particles), shape and structure may be used in close association with cells or drugs or combinations thereof to, for example, facilitate their delivery or enhance their effectiveness as therapeutic agents. Cells of particular interest include stem cells and other progenitor cells. Drugs of particular interest include a variety of substances having potential therapeutic value, such as various cytokines, hormones, growth factors and small molecule pharmacological agents.

Particles may include woven or non-woven polymeric meshes or other porous or nonporous structures suitable, for example, for immobilizing viable cells and polymeric or other materials used, for example, to encapsulate drugs, cells or both. Drugs of high molecular weight may also be considered particles or, more particularly, nano particles. Cells, drugs and particles or combinations thereof are often dispersed in a delivery medium to promote or preserve their viability or efficacy or to facilitate their delivery. Examples of typical delivery media include normal saline (0.9% NaCl solution) or Dulbecco's Modified Eagle Medium (“DMEM”), but could also include certain polymeric materials such as polyvinyl alcohol.

In many circumstances, it may be advantageous to use another substance or other substances in close association with (or in combination with) a therapeutic agent or agents. Close association of a substance and a therapeutic agent or multiple agents could involve mixing or other means of dispersion, physical interaction(s), chemical interaction(s) or various combinations thereof. An example of a particularly simple close association between substance and therapeutic agent or agents is mixing or otherwise dispersing the therapeutic agent(s) in, for example, a fluidic delivery medium. The delivery medium is immersive surrounding the particles, drugs or combinations thereof and thereby serves to facilitate their delivery.

An example of a suitable fluidic delivery medium relevant to using cells as therapeutic agents is DMEM. DMEM is well recognized as effective in maintaining cell viability. The fluidic delivery medium also physically supports and protects the cells and is therefore effective in facilitating their delivery. In addition to cells, other therapeutic agents such as drugs or particles or combinations thereof can be similarly mixed or otherwise dispersed in fluidic delivery media such as normal saline or DMEM.

Yet another example of close association involving, in this case, physical interaction, is encapsulation of the therapeutic agent or agents in a substance. Encapsulating substances may be permeable to the therapeutic agent or not and may also be designed to exhibit certain properties. For example, encapsulating substances can be selectively permeable to substances produced by or used by cells. Capsules may also preferentially chemically, mechanically or otherwise bind to certain surfaces in ways that enhance the therapeutic value of the encapsulated therapeutic agent(s). Examples of encapsulation include cells in more or less hollow, porous spheres made of algenate or drugs contained in nano tubes.

The close association of a therapeutic agent with other substances can be advantageous. For example, the combination could act to enhance the therapeutic value of the therapeutic agent or agents, facilitate their delivery or produce some other desirable characteristic that may be lacking in the therapeutic agent or substance alone. For example, encapsulation can enhance the efficacy of a therapeutic agent or agents by slowly releasing the encapsulated drug or drugs. In other examples, cells immersed in DMEM can exhibit enhanced viability, and encapsulation shields a therapeutic agent or agents from exposure to the biomechanical forces normally associated with delivery. These forces may be detrimental to the effectiveness of the therapeutic agent or agents.

Accordingly and in view of the aforementioned examples, the close association of a substance with a therapeutic agent or agents may result in the formation of particles of varying size, shape, material, structure and biological or other function. The foregoing examples of close association are not exhaustive, and additional examples would be apparent to one skilled in the art having benefit of this disclosure.

Delivery means or devices, such as needles, catheters or systems comprising needles, catheters and other devices, can deliver cells or drugs or particles or combinations thereof, together with their associated substances or carriers (if present). Such means exert biomechanical forces during the delivery process, forces that can in various ways affect the efficacy of the therapeutic agent or agents being delivered. Examples of biomechanical forces include flow induced shear stresses and interfacial forces exerted between cells, drugs or particles in close proximity to surfaces or to one another. Shear stress may be particularly deleterious to a therapeutic agent when flow in a delivery means or device or system exceeds certain limits causing, for example, cell lyses or death. Interfacial forces can also be excessive, for example, when the volume fraction of cells, drugs or particles in a delivery medium is too high. Studying effects of biomechanical forces on various therapeutic agents, particularly cells, is an active area of medical investigation, with literature on the subject including data.

Moreover, environmental factors associated with processing and handling of therapeutic agents, such as temperature, relative humidity, light intensity and spectral character and pressure can impact efficacy of agents and their associated substances.

There is a rapidly growing body of available data showing the value to human health of therapeutic agents that are sensitive to delivery, handling, and/or environmental factors, with a representative few such agents being discussed above. As discussed in further detail below, certain exemplary embodiments of the present invention can comprise a process and/or a system for optimizing the effectiveness of cell, drug or particle based therapies, for appropriately advising interventionalists and/or for otherwise guiding or controlling agent delivery. Such an embodiment can provide a systematic means for maintaining therapeutic efficacy in an environment of biomechanical forces and other factors known to influence cells, drugs and particles as therapeutic agents.

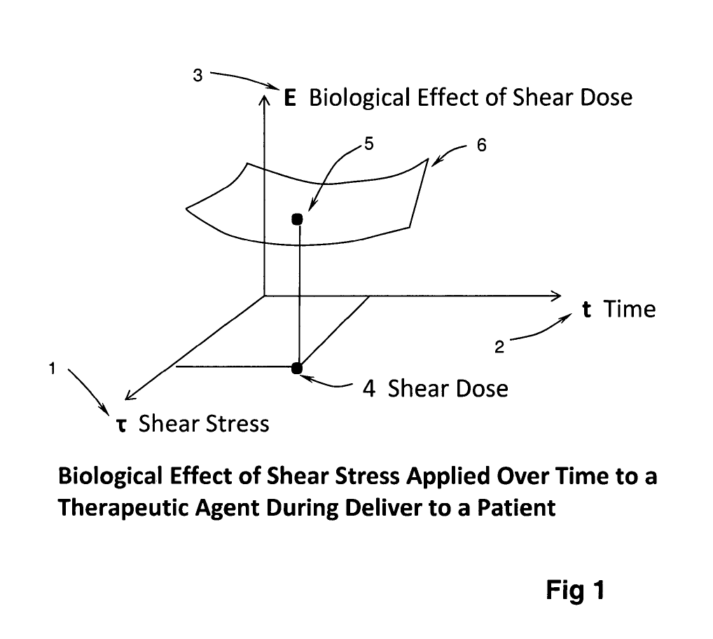

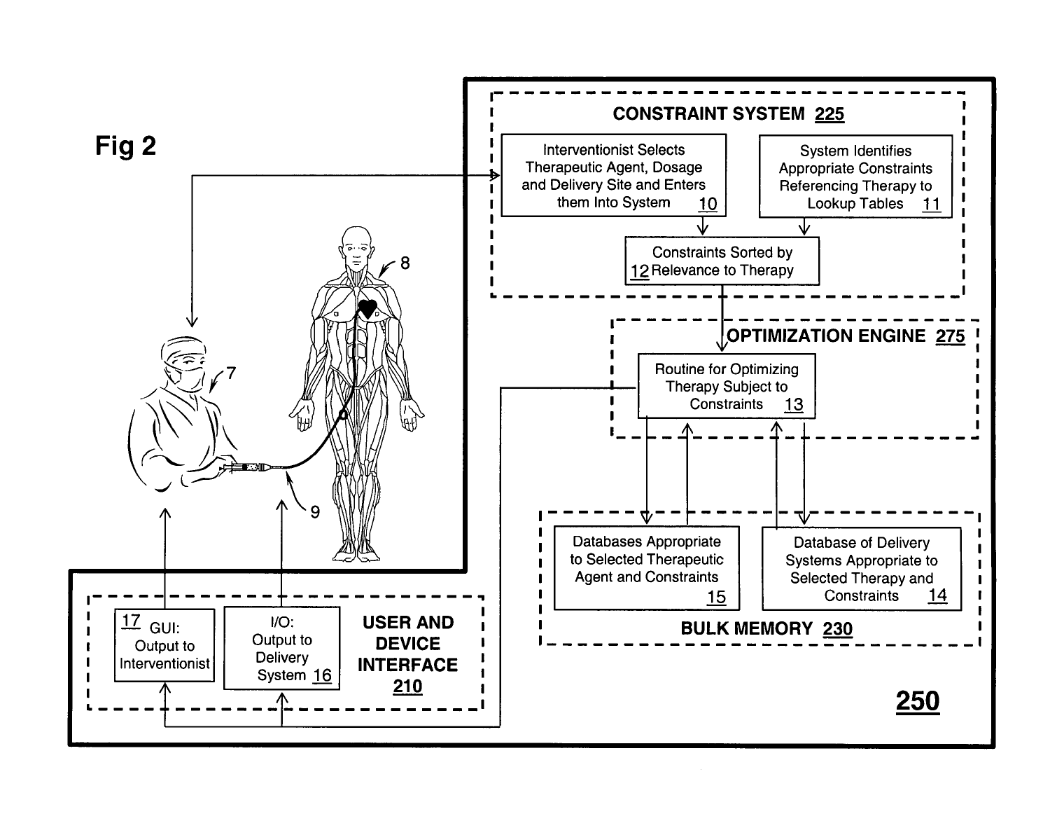

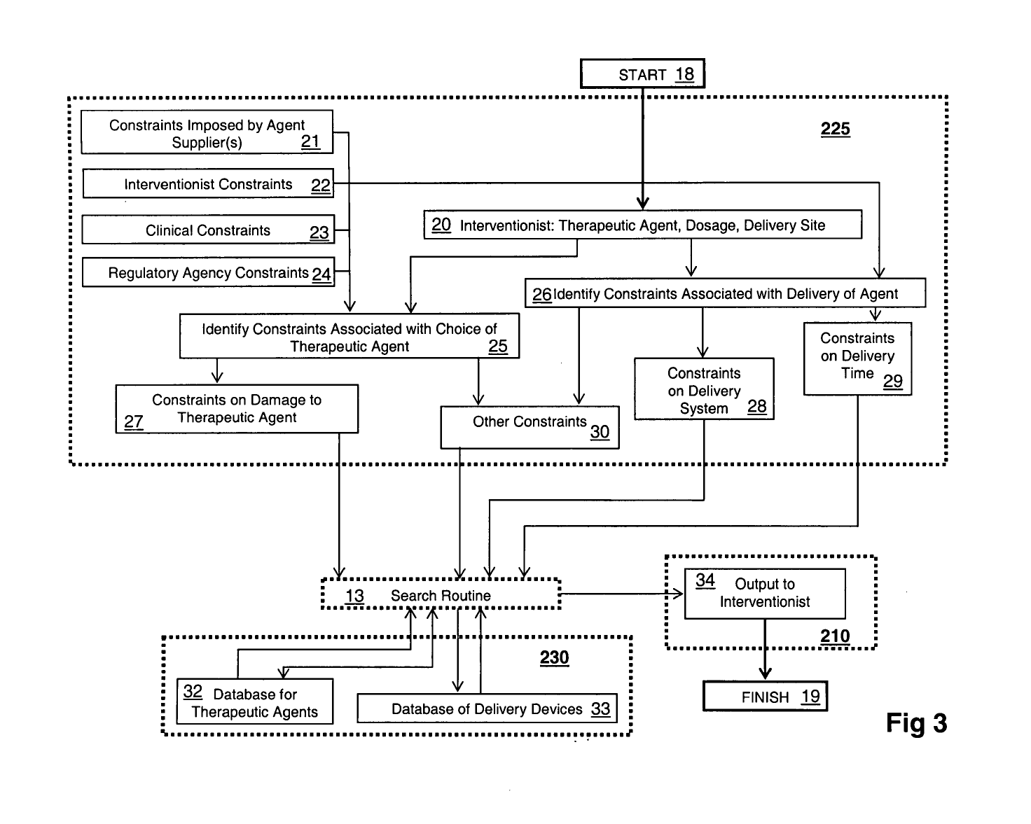

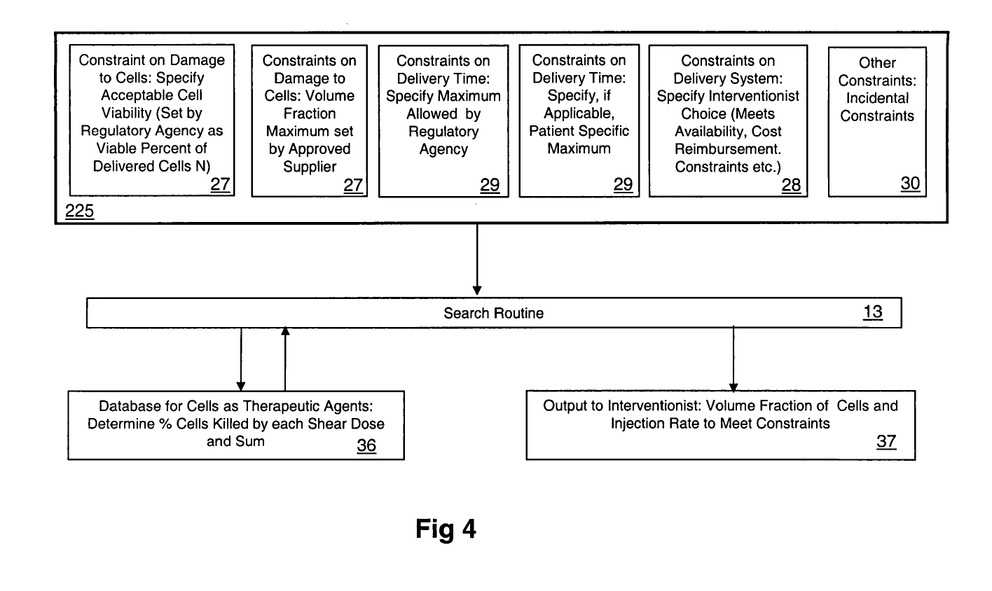

The present invention will be discussed more fully hereinafter with reference to FIGS. 1-6, which provide additional information regarding representative or illustrative embodiments of the present invention. FIG. 1 graphically illustrates therapeutic effectiveness as a multidimensional function of applied biomechanical forces. FIG. 2 illustrates an exemplary system for managing delivery of a therapeutic agent to avoid a situation whereby biomechanical forces cause unwanted effects or diminished therapeutic effect. FIG. 3 illustrates an exemplary process for the system of FIG. 2. FIGS. 4-6 illustrate a representative example for operating the system illustrated in FIG. 2 via the process of FIG. 3.

In an exemplary embodiment of the present invention, an interventionalist specifies certain parameters of a medical intervention that involves delivering a therapeutic agent, while leaving other parameters of the intervention open. The therapeutic agent is susceptible to biomechanical forces associated with delivery, and the open parameters can influence the efficacy of the agent and thus the therapeutic outcome of the intervention. A computer-based system narrows the range of open parameters (or constrains the open parameters) via referencing the interventionalist-specified parameters to one or more databases. From the narrowed ranges, an optimization routine identifies suitable values for the open parameters for conducting the intervention. The identified values avoid unwanted change of the therapeutic agent due to biomechanical forces or other delivery-related influences.

The present invention can be embodied in many different forms and should not be construed as limited to the embodiments set forth herein; rather, these embodiments are provided so that this disclosure will be thorough and complete, and will fully convey the scope of the invention to those having ordinary skill in the art. Furthermore, all “examples,” “embodiments” and “exemplary embodiments” given herein are intended to be non-limiting, and among others supported by representations of the present invention.

This document includes sentences, paragraphs, and passages (some of which might be viewed as lists) disclosing alternative components, elements, features, functionalities, usages, operations, steps etc. for various embodiments of the present invention. Unless clearly and explicitly stated otherwise, all such lists, sentences, paragraphs, passages and other disclosures are not exhaustive, are not limiting, are provided in the context of describing representative examples and variations, and are among others supported by various embodiments of the present invention. Accordingly, those of ordinary skill in the art having benefit of this disclosure will appreciate that the present invention is not constrained by any such lists, examples, or alternatives. Moreover, the inclusion of lists, examples, embodiments and the like will help guide those of ordinary skill in practicing many more implementations and instances of the present invention without undue experimentation, all of which are intended to be within the scope of the claims.

This disclosure includes figures and discussion in which features and elements of certain embodiments have been organized into functional blocks, subsystems or modules. And certain processes and methods have been organized into steps. Such organization is intended to enhance readership and to teach the reader about working principles of the present invention and about making and using an abundance of embodiments of the present invention. The organization is not intended to imply or force any rigid divisions or partitions that would limit the present invention. In practice, functionalities, elements, steps and features can be dispersed or grouped differently. The inclusion of an element or function in one block, module, or subsystem verses another will be substantially arbitrary in many instances, with the divisions being soft and readily redrawn. Likewise, the inclusion of one action in one step rather than another is often substantially arbitrary. Accordingly, functional blocks, modules, subsystems and the like can be combined, divided, repartitioned, redrawn, moved or otherwise altered without deviating from the scope and spirit of the present invention.

In view of the foregoing, it is desirable to quantify or evaluate biologically meaningful effects of a biomechanical force or forces or an environmental or other factor or, more generally, a combination of forces or factors on a therapeutic agent or agents or on an agent or agents closely associated with another substance. A Cartesian coordinate system is one way (but not the only way) to represent relevant data. For example, relevant data can also be tabulated or stored in a searchable database. FIG. 1 illustrates a Cartesian system wherein the coordinate axes are labeled 1, 2 and 3. These axes can be used to represent or graphically or functionally describe any two- or three-dimensional data obtained from experiments, models or combinations thereof.

Those of ordinary skill having benefit of this disclosure will appreciate that the three-dimensional “space” shown in FIG. 1 is exemplary and that many cases may involve an n-dimensional space. Thus, the coordinate system might have four, five, eight, ten, twenty or fifty axes, for example.

By way of example, the maximum, mean or other relevant measure of the fluid shear stress τ to which a fluidic media containing a therapeutic agent is exposed is plotted on axis 1. The duration of exposure to the shear stress τ is the time t plotted on axis 2. The point 4 in the τ−t plane in FIG. 1 represents a “shear dose” to which the therapeutic agent is subjected. Axis 3 in FIG. 1 represents any measurable and relevant biological effect E of a given shear dose on a particular therapeutic agent. The effect E of a given shear dose may be strongly or weakly dependent on the magnitude of the shear dose and could even, for a range of time t, be independent of shear dose. In the latter case, it is the shear stress that determines the magnitude of the biological effect E.

A therapeutic agent may be subjected sequentially to multiple shear doses during delivery even when delivery rate is constant. This may occur, for example, when using a delivery system combining a catheter (producing one shear dose) and a needle (producing a second dose). The effect or effects of multiple shear doses could be complex and may be represented using multiple data plots rather than a single data plot. For example, shear dose effects could be independent, cumulative or multiplicative, or none or all of these could apply. FIG. 1 illustrates a representative case of a single shear dose having a single biological relevant effect E.

In the more general, n-dimensional case discussed above, additional factors may influence the effect of a shear dose. Such factors might include environmental parameters, temperature, volume ratio, etc. In this situation, a three-, four-, five-, or ten-dimensional vector or space (corresponding to the τ−t plane) can produce the biological effect E represented on the axis 3. Also, the biological effect E may itself comprise a multidimensional result. Such a multidimensional result might comprise a range or population of phenotypes in a set of cells subjected to conditions represented on the τ−t plane (or a multidimensional space).

Point 5 of FIG. 1 is a biologically relevant effect E corresponding to shear dose 4. Multiple measurable effects E are known consequences of exposure of therapeutic agents to shear stress τ of duration t. Many (but not necessarily all) such effects may be documented in the literature or otherwise known or understood. Thus, the present system and method accommodates expanded knowledge resulting from experimentation, research, heuristic and public knowledge.

For example, in the case of cells as the therapeutic agent, E could be the percent of viable cells or the number of cells with a given phenotype remaining after exposure to a specified shear dose. The potential therapeutic value of cells, drugs, particles or combinations thereof and the growing investment in research and development aimed at utilizing them as routine and effective therapeutic agents, means that more measurable effects E are becoming known, understood, and/or emerging. Newly identified effects can be readily expressed in plots like FIG. 1 or individually or collectively incorporated into databases.

The point 5 is the value of the biologically relevant effect E for shear dose point 4. In some instances, points 5 may be known for multiple but not all possible shear doses 4. The surface 6 shown in FIG. 1 may represent a mathematical best fit to all known data points 5. Some variability in E (e.g. points 5) for the same shear dose 4 is expected. This variability could, for example, be a manifestation of using varying volume fractions of the therapeutic agent in the delivery media in the determination of E. In this case, data points 5 may be alternatively characterized by multiple surfaces or by a point cloud of data.

Although other factors may be relevant, a connection between volume fraction and variability of the data is expected. As the volume fraction of the therapeutic agent increases, increased numbers and intensities of mechanical interactions between cells or particles or drugs with each other or with surfaces can occur. These forces can have an additional biologically relevant effect on the therapeutic agent(s). For example, closely packed cells (in a high volume fraction) could have their gene expression or phenotype changed in ways not seen when cells are exposed to the same shear dose but at a lower volume fraction of cells in fluidic media.

FIG. 2 illustrates an exemplary system 250 that guides, manages or advises delivery of a therapeutic agent to avoid subjecting the therapeutic agent to a level of biomechanical forces that might negatively impact therapeutic effectiveness according to certain embodiments of the present invention. FIG. 2 further illustrates an example of an overall way the system 250 and certain of its exemplary components 10-17 work with a patient 8 and an interventionalist 7 (or some other physician, practitioner, medical practitioner, surgeon, doctor, nurse, assistant, operator, technician, person or delivery device) to deliver, in this case to the patient's heart, a therapeutic agent using a delivery device or system 9. The delivery device 9 could, for example, be either a catheter, cannula, needle, endoscope channel or other tube extending into the patient 8 or a delivery system comprising, for example, a combination of one or more such devices. In various exemplary embodiments, the delivery device 9 can comprise venous infusion catheter such as a peripheral infusion catheter and or central venous infusion lines. Also, arterial infusion catheters such as thrombolytic infusion catheters and angiographic catheters and various diagnostic and infusion catheters for other peripheral, central and cranial applications. In many exemplary embodiments, the delivery device 9 comprises a flow restriction, a bend, or a change in internal cross section (e.g. form, geometric shape, or size) that produces shear stress on the therapeutic agent.

In an exemplary embodiment, the system 250 comprises a computer or other machine that can execute instructions, including a processor, input and output system, keyboard or other user input device, graphical user interface (“GUI”), etc. For example, the system 250 can comprise a special purpose computer, an embedded system, an embedded controller, a personal computer, or a laptop, for example. More specifically, the system 250 can implement appropriate steps of the processes, methods and routines discussed below.

The system 250 can connect to, comprise, or otherwise access assorted types of memory that may hold instructions. Such memory can include any one or combination of volatile memory elements (e.g., forms of RAM such as DRAM, SRAM, SDRAM, etc.), nonvolatile memory elements (e.g., ROM, hard drive, tape, compact disc read-only memory (“CDROM”), etc.), and erasable memory (e.g. erasable programmable read only memory (“EPROM”) and electrical EPROM (“EEPROM”)). Moreover, the system 250 may incorporate electronic, magnetic, optical, and/or other types of storage media and can have a distributed architecture, where various components are situated remote from one another, but can be accessed over a network. Instructions and/or code for implementing the methods, processes and routines discussed below can be stored in a computer-readable medium.

A “computer-readable medium” can be any means that can store, communicate, propagate or transport a program for use by or in connection with an instruction execution system, apparatus, or device. The computer readable medium can be, for example but not limited to, an electronic, magnetic, optical, electromagnetic, infrared or semiconductor system, apparatus, device or propagation medium. More specific examples (a non-exhaustive list) of the computer-readable medium can include the following: an electrical connection (electronic) having one or more wires, a portable computer diskette (magnetic), a RAM (electronic), a read-only memory (“ROM”) (electronic), an erasable programmable read-only memory (EPROM, EEPROM, or Flash memory) (electronic), a data stick, a flash drive and a portable CDROM (optical). Note that a computer-readable medium could even be paper or another suitable medium upon which the program is printed, as the program can be electronically captured, via for instance optically scanning the paper or other medium, then compiled, interpreted or otherwise processed in a suitable manner if necessary, and then stored in a computer memory.

In certain exemplary embodiments, the methods, processes and routines disclosed herein can include logic implemented in hardware with any or a combination of the following technologies: a discrete logic circuit(s) having logic gates for implementing logic functions upon data signals, an application-specific integrated circuit (“ASIC”) having appropriate combinational logic gates, a programmable gate array(s) (“PGA”), a field programmable gate array (“FPGA”), etc.

It should be apparent that one of ordinary skill in the art would be able to make and operate the methods, processes and routines disclosed herein without difficulty and without undue experimentation based on the figures, illustrations, exemplary functional block diagrams, flow charts, and associated descriptions in the application text, for example. Therefore, additional disclosure of a particular set of program code instructions or more particularized circuit or logic schematics are not considered necessary for an adequate understanding of how to make and use the present invention.

Certain steps in the processes and methods described below (and elsewhere herein) may naturally need to precede others for the present invention to function as described. However, the present invention is not limited to the order of the steps described if such order or sequence does not adversely alter the functionality of the present invention to the extent of rendering the invention inoperable. That is, it is recognized that some steps may be performed before or after other steps or in parallel with other steps without departing from the scope and spirit of the present invention.

The system 225 comprises a constraint system 225 that manages applying constraints to potential delivery scenarios, thereby reducing the available delivery parameters to facilitate optimization or otherwise identifying preferred delivery conditions. In the exemplary embodiment of FIG. 2, the constraint system 225 comprises a module 10 through which an interventionalist 7 can select or otherwise identify a therapeutic agent, a dosage and delivery site (e.g. a region of a specific organ to be treated). The interventionalist 7 can make such inputs using a GUI 17, touchpad, voice input, or a keyboard, for example. The constraint system 225 further comprises a module 11 through which the system 250 identifies appropriate constraints via referencing a selected therapy to one or more lookup tables or other database, for example. The module 12 sorts the constraints according to relevancy to the therapy chosen by the interventionalist 7. In accordance with the example of FIG. 2, basic choices made by the interventionalist 7 are combined with constraints on the therapy available from multiple sources and sorted. An exemplary embodiment of the constraint system 225, including representative constrain management capabilities and actions of the modules 10, 11, and 12 will be described in further detail below with reference to FIGS. 3, 4, 5, and 6.

The system 250 comprises an optimization engine 275 for optimizing delivery of the therapeutic agent (and/or of a medical intervention that comprises delivering the therapeutic agent) to avoid unwanted change in therapeutic effectiveness stemming from biomechanical forces or other delivery influence. In an exemplary embodiment, the optimization engine 275 comprises a computer-implemented optimization routine 13 that optimizes according to constraints sorted by the constraint system 225. The optimizing routine 13 identifies, selects or determines one or more parameters for delivering the therapeutic agent in a way that avoids violating the constraints. An exemplary embodiment of the optimization engine 275, including representative capabilities and actions, will be described in further detail below with reference to FIGS. 3, 4, 5, and 6.

The system 250 also comprises a memory 230, such as one or more servers, hard drives, or optical storage systems. The memory 230 can be collocated with the optimization engine 275 and the constraint system 225. Alternatively, the memory 230 can connect to other elements of the system 250 via a communication link or a network, such as a local area network (“LAN”), a wide area network (“WAN”) or a satellite link, for example. The memory 230 contains databases 15 appropriate to selected therapeutic agents and to constraints and databases 14 of delivery systems appropriate to selected therapy and constraints. Thus, additional data is available to the optimization routine 13 from a database 14 for delivery devices and a database 15 containing information about therapeutic agents. As will be discussed in further detail below in the context of at least one specific example, the optimization engine 275 optimizes via referencing constraints form the constraint system 225 to the databases 14 and 15.

The system 250 comprises a user and device interface 210 that can provide output to either or both a person, such as the interventionalist 10, and an apparatus or machine, such as the delivery device 9. Output is used either to control the delivery device 9 or inform the interventionalist 17 delivering the agent. Thus, the system 250 can actuate mechanical or electrical devices that interface with or otherwise control the delivery device 9 via input/output (“I/O”) 16 in the form of current or voltage signals, for example. Via the GUI 17, the interventionalist 7 can input constraints or therapeutic selections and can receive output from the system 250. The system 250 can provide instructions, guidance, advice, recommendations or other information useful to the interventionalist through the GUI 17. Such information can be presented for visual reception by the interventionalist 7 or alternatively for auditory or tactile reception.

FIG. 3 is a block diagram showing a representative flow of data for an exemplary process 300 used to inform decision making and or otherwise influence or control delivery of therapeutic agents such as drugs, cells and particles.

The process 300 begins at step 18 labeled as “START” and ends at step 19 labeled “FINISH” in FIG. 3. At step 20, the interventionalist 7 identifies his or her choice of therapy via, for example, selecting the therapeutic agent, delivery site and the dosage to be used. In an exemplary embodiment, step 20 can be viewed as analogous to what is routinely done in traditional therapy where an interventionalist or physician makes conventional choices but uses traditional therapeutic agents such antibiotics and a delivery device or means such as intramuscular injection.

In the current scheme, the therapeutic agent can be cells, drugs or various combinations thereof either with or without closely associated substances. The therapeutic agent may be delivered in a substantially pure state or in a carrier or buffer, for example. Moreover, the volume fraction can vary from 100 percent downward, for example. The delivery site can be an organ, organs or an organ system in need of treatment and the dosage specifies the amount of therapeutic agent to be delivered. The dosage can, for example, be the number of viable cells of a specified phenotype to be delivered or the delivered weight of a large molecule drug.

Steps 21, 22, 23 and 24 represent typical sources for constraints that may or may not be placed on the process via the constraint system 225. Taken together, these steps generate a constraint profile for the selected therapy and its delivery to a patient.

At step 21, the system 250 applies supplier constraints, or supplier constraints are entered into the system 250. Suppliers of a particular therapeutic agent may impose constraints related to their business as suppliers and their need to meet standards set by agencies regulating suppliers. The system 250 imposes such constraints at step 21. Examples include availability of agents on certain time lines or in standard dosages, agent shelf life, and, possibly, limited options for the fluidic delivery media and for the volume fraction of the agent in that media. Agents may also be supplied in ways which limit further choices as, for example, by prepackaging them in conjunction with delivery means, devices or systems.

At step 22, the system 250 applies interventionalist constraints, or the interventionalist enters such constraints into the system 250. Typically, the module 10 of the constraint system 225 manages such interventionalist constraints. Exemplary interventionalist constraints include aspects of the therapy and of the particular patient being treated which are typically left to the discretion of the interventionalist. An example of a constraint set by an interventionalist is allowing only a favored delivery device or devices or system to be used to deliver the therapeutic agent. Other examples of constraints can include restrictions based on the interventionalist's best judgment of the patient's medical history and condition. These could include for example, choices related to dosage within a range of acceptable dosages apropos the patient's age, weight and medical status.

At step 23, the system 250 (typically the constraint system 225) applies clinical constraints, or such constraints are entered into the system 250. Exemplary clinical constraints are a consequence of (or may stem from) policies, procedures and business practices established by a particular clinic which can influence various aspects of the treatment processes. For example, restrictions related to what agents can be used, from which contract suppliers, insurance issues such as reimbursement and liability and therapy related overhead costs.

At step 24, the system 250 (typically the constraint system 225) applies regulatory agency constraints or such constraints are entered into the system 250. Exemplary regulatory agency constraints 24, can include rules, guidelines, criteria or other constraints set by the US Food and Drug Administration (“FDA”) or other regulatory body, organization, or government entity, for example. Regulatory agency constraints can relate primarily to clinical guidelines and regulations as might arise as part of a regulatory approval process for a particular therapy, for example. Such guidelines and regulations may be subsequently issued to users and potential users of the therapy by the regulatory agency. Regulatory guidelines could, for example, specify a range of dosages, acceptable delivery times and other details regarding the therapeutic agent as handled, preserved and delivered. In the case of cells as the therapeutic agent, the number of viable cells or the number of cells of a particular phenotype could, for example, be specified.

At steps 25 and 26, input from steps 21, 22, 23, and 24 is sorted and divided into two categories. Typically, the module 12 of the constraint system 225 implements the sorting and dividing via executing software or code. Constrains related to the choice of the therapeutic agent are identified at step 25 while constraints related to delivery of the agent are identified at step 26. At step 27, the selected therapeutic agent is associated with an acceptable level of damage to that agent. For example, damage modes, damage measures and tolerances for each measure can be selected from databases (not shown in the Figure) for a given agent. For example, for cells as the therapeutic agent of choice, at step 27 the system 250 could identify the shear stress exerted upon delivery as the damage mode, the damage measure could be the percentage of cells used that are viable post delivery and the tolerance on this measure could be set by a regulatory agency such as the FDA as the minimum percent of delivered cells that must be viable.

While substantial data exists for cell death due to shear stress, additional or supplemental data may be used for measuring more subtle effects, such as the effects of shear stress on the phenotype of delivered cells. Damage modes, measures and tolerances can also be used for other therapeutic agents and for these agents in combinations with their closely associated substances.

At step 28 the system 250 identifies constraints on the delivery means, device or system. These could simply indicate availability, for example, or devices, systems or configurations of multiple devices or means favored by the interventionalist. Constraints could also be limitations placed on available delivery devices or systems by the clinic reflecting cost, reimbursement issues or availability of trained support staff, for example.

Step 29 represents application of acceptable delivery times for a selected therapeutic agent. These may come from a regulatory stipulation or from patient specific situations. At step 30, the system 250 may apply other constraints as may be unique to a particular situation or a particular therapeutic agent. For example, additional constraints may be applied that avoid neatly fitting into the other illustrated categories. Examples include a catalog of difficulties observed when using certain devices, agents, techniques or suppliers or notes about successes intended to inform future users of a particular device, agent or approach.

Step 13 reflects a search routine. In an exemplary embodiment, the search routine is a constrained optimization of a multidimensional or complex system with multiple input variables and constraints. Its functions are, for example, advising the interventionalist or controlling the delivery of an agent or agents or otherwise assist those using drugs, cells or particles as therapeutic agents. An exemplary scheme for performing such an optimization will be described in further detail below. Other schemes will be apparent to those of ordinary skill having benefit of this disclosure and are readily given for other examples.

In addition to input from steps 27-30, the search routine 13 also uses data from the database for therapeutic agents 32, which can be an embodiment of the database 15 illustrated in FIG. 2 and discussed above. The database 32 (and/or the database 15) contains results from experiments and modeling of the effects of biomechanical forces, environmental factors and the like and in combination on specific therapeutic agents. FIG. 1 is representative of the kind of data available for execution of the search routine at step 13, to implement delivery optimization. The database of delivery devices 33, which can be an embodiment of the database 14 illustrated in FIG. 2 and discussed above, contains available information on approved and marketed delivery means, devices or systems including, for example, dimensions, costs, materials used and general availability. Accordingly, the search routine 13 draws from the databases 32 and 33 for delivery optimization.

At step 34, the output of the search routine 13 is compiled into a format useful to and readily understood by the interventionalist 7 and/or others charged with administering the therapy. A graphical format can be effective for this purpose, as supported by the GUI 17 illustrated in FIG. 2 and discussed above. The output could also, for example, inform or control the delivery device 9 or other device set up to infuse a therapeutic agent in compliance with the imposed constraints.

Turning now to FIGS. 4-6, a representative example will be described to further illustrate an exemplary data flow and an exemplary embodiment of the search routine 13 illustrated in FIG. 3 and discussed above. Examples of representative input data can include critical constraints without including all the input data that may be applicable or used. The embodiment of FIG. 4 includes specification of other constraints 30 that can come from various sources, are known and available but may be incidental to the process. Examples of other constraints include, for example, constraints on scheduling a procedure which is typically an incidental rather than critical constraint. FIG. 4 illustrates additional incidental data or constraints that will lead one skilled in the art to providing a tailored set of constraint appropriate for particular circumstances and applications.

At step 20, the interventionalist 7 selects his or her therapy of choice by choosing the therapeutic agent, the dosage and the delivery site. For example, the interventionalist 7 might elect to deliver a dosage of ten million viable embryonic stem cells (an exemplary agent) to a specified region of a heart infarct (an exemplary delivery site). In this example, an additional constraint is added by the interventionalist 7. Specifically, at step 22, the interventionalist 7 specifies a catheter-based delivery system with which the interventionalist 7 is familiar and well trained. The delivery device 9 selected is available, reimbursable and meets all other incidental constraints imposed by the clinic (step 23) and regulatory agencies (step 24).

The stem cells are available from a supplier who meets all regulatory agency stipulations governing suppliers of stem cell based therapies (step 21). The cells are available at user specified volume fractions of up to seventy percent cells and only in DMEM so as to limit cell damage in handling (steps 21 and 24).

In order to eliminate environmental and transport damage, cells are delivered by the supplier within one hour of a request from the clinic (step 21). The specific clinical case involves a patient who, for interventionalist-specified reasons (step 22) or clinic-specified reasons (step 23), cannot undergo a delivery procedure lasting longer than, for example, five minutes. The regulatory agency specifies various incidental handling procedures for the supplier, clinic and interventionalist. The agency also specifies critical constraints such as the percent of the delivered cells which must be viable and the maximum time available for delivery of the cells using an approved delivery device or system (step 24). As illustrated in FIG. 4, the constraint system 225 can comprise various constraint modules 27, 28, 29, and 30.

In this example, the biologically relevant effect E is cell viability. The viability of the stem cells is known to depend on shear dose and cell volume fraction as might be characterized graphically by FIG. 1 or as an appropriate database. The range of shear doses producing acceptable viability is known from this data. At a given shear dose, cell death inducing damage is more likely to occur at higher cell volume fraction than at lower cell volume fraction. This can happen because higher cell volume fractions may result in shorter delivery times but possibly additional cell death due to potentially higher viscosity and hence shear dose and also denser packing of cells. Lower cell volume fractions result in longer delivery times but less cell death at any shear dose. Hence an optimization is useful.

For this example, the database for the therapeutic agent 32 becomes specifically a database for cells as therapeutic agents 36. In other words, the database 36 can be a specific embodiment of the database 32. The database 36 contains experimental and modeling data on the effect of shear dose on the viability of cells immersed, as selected here, in DMEM at a specified volume fraction. The output provided by the user and device interface 210 to the interventionalist 7 is, for this example, an output specifying the cell volume fraction and injection rate to meet the constraints specified (step 37). The output at step 37 can also be directly coupled to a delivery system, such as the delivery device 9, a pump or some other machine and used to appropriately control the delivery process. The information relevant to the selected delivery system needed for the search (optimization) routine 13 is available from the database of delivery systems 33 (see FIG. 3).

Additional examples may be constructed by adding or removing constraints. For example, adding the effect of shear stress on cell phenotype may involve additional data on the effect of shear stress or shear dose or cumulative shear dose and volume fraction of cells on cell phenotype. Such information can be ascertained by one of ordinary skill in the art having benefit for this disclosure and without undue experimentation, for example via routine laboratory instrumentation and techniques.

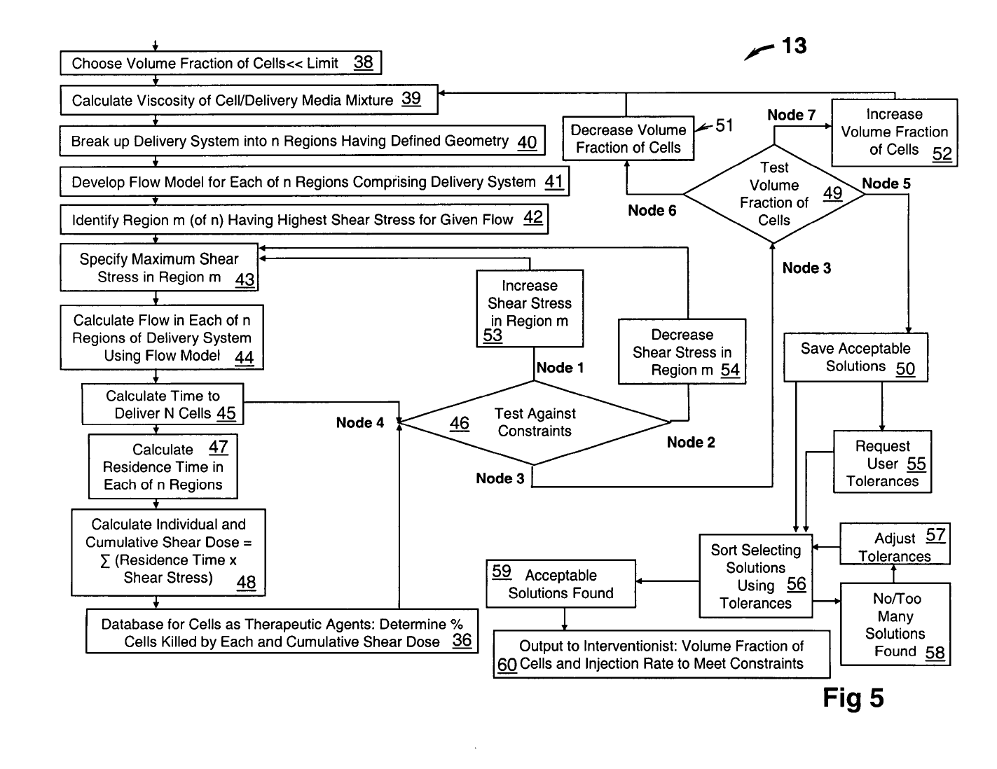

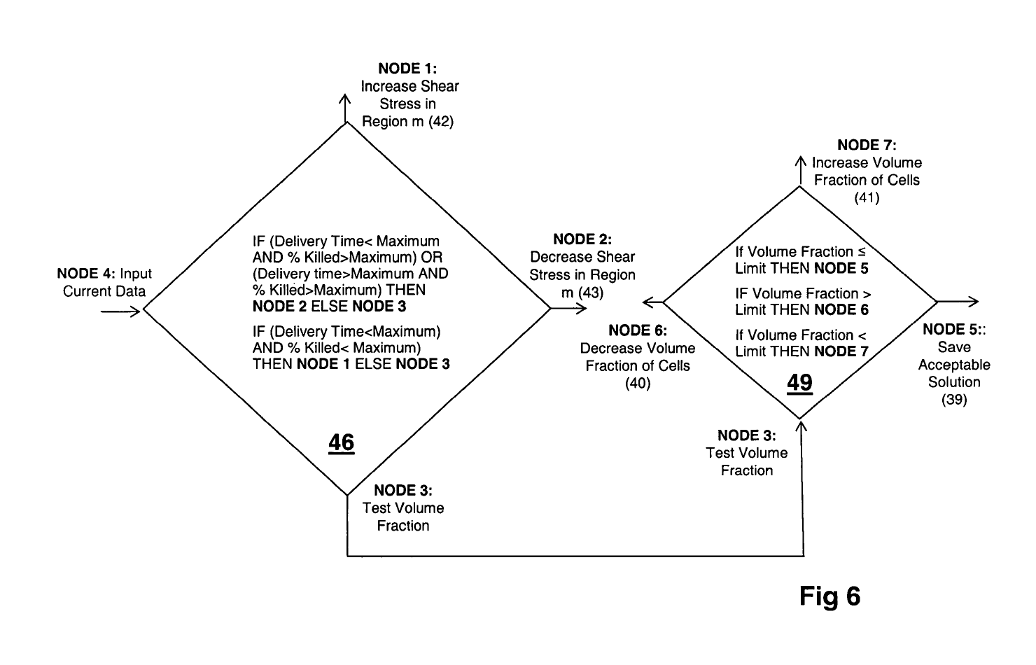

An exemplary embodiment of the search routine 13 illustrated in FIGS. 3 and 4 for the current example is shown in expanded form in FIGS. 5 and 6. To begin, a volume fraction of cells is selected at step 38 that is much smaller than the previously specified maximum (see step 27 on FIG. 4). The viscosity of the cells in DMEM is calculated at step 39 using, for example, a power law or other suitable law characterizing the effect of cell volume fraction on the viscosity of the combination of cells and media.

The selected delivery means, device, or system is then broken into n regions at step 40, each having a geometry which is fixed and well characterized. This breakup facilitates developing a flow model for the delivery device 9 at 41. The flow model can take many forms. For example, simple steady viscous flow in a tube, as in Poiseulle flow, or more complex models based on computational fluid mechanics can be used to construct the model. Such models are readily understood and can be developed by those skilled in the art having benefit of this disclosure without undue experimentation.

With the flow model established, the region m of the n regions constituting the delivery means, device or system is selected to be the region where a shear stress measure is a maximum at step 42. In certain exemplary embodiments, shear stress can be elevated at a tight bend, a corner, a change in cross sectional shape or size or in connection with an exit or entrance port, for example. The model can compute shear stress due to such features, for example. The shear stress measure could be the mean or peak shear stress or some other measure. The measure chosen usually should provide the best correlation with cell damage or death. Typically, the peak shear stress serves this need well but other measures could also be used. For example, peak or mean or other measure of shear rate could be relevant. Adapting the illustrated embodiments to practice this variation is readily accomplished those ordinarily skilled in the art having benefit of this disclosure and can be implemented without undue experimentation.

A shear stress measure in region m is selected at step 43. The value chosen typically should be much smaller than any value known from the database 36 to cause cell death. This choice accommodates an iterative process for performing the constrained optimization. The shear stress in each region of the delivery device 9 can be calculated at step 44 using the flow model developed at step 41 and recognizing that the flow is the same in each of the n regions of the delivery device 9.

From the known flow developed at step 41, volume fraction (step 38) and number of cells to be delivered the time needed to deliver the specified number of cells (step 20) can be calculated at step 45. This is part of the information for testing against constraints (node 4 of step 46). Next, the times that the cells in DMEM spend in each of the n regions of the delivery system are calculated at step 47. The individual and cumulative shear dose is then calculated at step 48 and used to search the database 36. This database 36 can be a component of the broader database 32. The search returns the percent of the delivered cells that are killed by the shear stress exposure following from the selected delivery rate for the selected delivery means, device or system and the selected cell volume fraction in DMEM. This information is also available for testing against the constraints (see node 4 of step 46).

An exemplary embodiment of testing at step 46 is shown in detail in FIG. 6 where the input from node 4, the known constraints and previously determined process parameters are used to make one of three possible decisions. Specifically, the three exemplary decisions are: 1) increase shear stress and hence flow in region m (see node 1); 2) decrease the shear stress and hence flow in region m (node 2); or 3) proceed to test the volume fraction against constraints (see node 3 and, subsequently, step 49). The process branches to node 1 if 1) the delivery time and the percent of the cells that are killed are both less than their respective maxima or 2) the delivery time is larger than its prescribed maxima and, at the same time, the percent of the cells killed is less than its prescribed maximum. For conditions other than these, the process proceeds to node 3. Similarly, the process branches to node 2 if the delivery time is less than its prescribed maxima and the percent of cells killed is larger than its specified maxima. For conditions other than these, the process branches to node 3.

Testing at step 49 compares the current value for the volume fraction of cells with the constrained maximum value and branches accordingly to decrease (see node 6) or increase (see node 7) the volume fraction. If the volume fraction is below its maximum value, the state is saved as acceptable (see node 5). Solutions saved at step 50 have met, within acceptable limits, either the constraint on the maximum delivery time or on the percent of cells killed using the parameters characterizing the current delivery process.

The path following nodes 6 or 7 leads to either decreasing (at step 51) or increasing (at step 52) respectively the value of the volume fraction and restarting the process at step 39. The path following nodes 1 and 2 leads to increasing (at step 53) and decreasing (at step 54) respectively the shear stress in the critical region m and restarting the process at step 43.

At step 55, additional input is requested from the user (typically the interventionalist 7 or some other individual actively involved in delivering the therapeutic agent) in the form of acceptable tolerances on the solution. If these are set too tightly, few if any solutions will be found. If set too loosely, too many solutions may be found. Solutions are sorted at step 56 and the tolerances applied to determine if acceptable solutions can be found. The user then has the opportunity to adjust tolerances at step 57 if an unacceptably large number of solutions are found or if no solutions are found at step 58. At step 59, an acceptable solution or solutions is found and is delivered to the interventionalist at step 60 for his or her use or for use as input to a device or devices controlling the delivery mode, system or device.

An exemplary method and system for guiding delivery of a therapeutic agent to avoid compromising the agent's healing power has been described in detail such that one of ordinary skill can make, use, and practice the present invention readily and without undue experimentation.

Furthermore, certain exemplary embodiments of the present invention can comprise a method for promoting efficacy of a therapeutic agent whose efficacy varies with shear stress incurred during delivery, the method comprising the steps of: receiving operator input specifying a parameter relevant to delivering the therapeutic agent; based on the received input, accessing from a database data that is relevant to the efficacy; in response to processing the accessed data with a computing routine, making a determination about another parameter relevant to the efficacy; and outputting a message for operator receipt according to the made determination. In accordance with certain ones of such embodiments, the operator input specifies a type of catheter selected by an operator, the accessed data describes a dimension of the specified catheter, processing the accessed data comprises computing shear stress, associated with moving the therapeutic agent through the catheter, based on a shear stress model, and the message comprises a recommendation about delivering the therapeutic agent with the specified catheter. In accordance with certain ones of such embodiments, the efficacy of the therapeutic agent varies with shear stress more than ordinary blood cells vary with shear stress.

Certain exemplary embodiments of the present invention can comprise a method for helping a medical practitioner make decisions about delivering a therapeutic agent whose effectiveness can be impacted by biomechanical force incurred during delivery, the method comprising the steps of: receiving input from the medical practitioner, the input making at least one decision and leaving other decisions open; computing the biomechanical force for a plurality of decision scenarios, each comprising a plurality of the open decisions; for each of the plurality of decision scenarios, computing an estimate of therapeutic effectiveness according to the computed biomechanical force; and based on the computed estimates, providing computer output for the medical practitioner regarding the open decisions. In accordance with certain ones of such embodiments, the therapeutic agent is more sensitive to the biomechanical force than blood or blood plasma.

Certain exemplary embodiments of the present invention can comprise a system for planning a therapeutic session that comprises delivering a therapeutic agent whose efficacy is susceptible to force applied during delivery, the system comprising: an input device operative to receive information about the therapeutic session from an operator; a computing module, in communication with the input device, operative to: compute the force for each of a plurality of therapy regimes according to the received information; for each of the therapy regimes, evaluate efficacy of the therapeutic agent according to the computed force; and responsive to the evaluation, select one of the therapy regimes over another of the therapy regimes; and an output device, in communication with the computing module, operative to output notification of the selected therapeutic regime to the operator for the therapeutic session. In accordance with certain ones of such embodiments, the therapeutic agent is more susceptible to the force than white blood cells or red blood cells are susceptible to the force.

Technology useful for promoting effectiveness of therapeutic agents that are sensitive to biological forces associated with delivery to a patient has been described in detail. From the description, it will be appreciated that an embodiment of the present invention overcomes limitations of the prior art. Those skilled in the art will appreciate that the present invention is not limited to any specifically discussed application or implementation and that the embodiments described herein are illustrative and not restrictive. Furthermore, the particular features, structures or characteristics that are disclosed may be combined in any suitable manner in one or more embodiments based on this disclosure and ordinary skill. Those of ordinary skill having benefit of this disclosure can make, use, and practice a wide range of embodiments via combining the disclosed features and elements in many permutations without undue experimentation. This disclosure not only includes the illustrated and described embodiments, but also provides a rich and detailed roadmap for creating many additional embodiments using the various disclosed technologies, elements, features, and their equivalents. From the description of the exemplary embodiments, equivalents of the elements shown herein will suggest themselves to those skilled in the art, and ways of constructing other embodiments of the present invention will appear to practitioners of the art. Therefore, the scope of the present invention is to be limited only by the accompanying claims.

What is claimed is:

1. A method for delivering stem cells or progenitor cells to biological tissue, the method comprising steps of:
determining one or more biomechanical forces on the stem cells or the progenitor cells associated with delivery of the stem cells or the progenitor cells;
assessing a sensitivity of the stem cells or the progenitor cells to one or more of the biomechanical forces; and
controlling parameters for delivery of the stem cells or the progenitor cells based upon the assessed sensitivity.

2. The method of claim 1, further comprising a step of specifying a device for controlling delivery of the stem cells or the progenitor cells.

3. The method of claim 1, further comprising a step of communicating the parameters for delivery of the stem cells or the progenitor cells to a device for controlling delivery of the stem cells or the progenitor cells.

4. The method of claim 1, wherein determining one or more biomechanical forces on the stem cells or the progenitor cells comprises evaluating a flow model of the stem cells or the progenitor cells within a device for controlling delivery of the stem cells or the progenitor cells.

5. The method of claim 1, wherein assessing the sensitivity of the stem cells or the progenitor cells to one or more of the biomechanical forces comprises evaluating the therapeutic efficacy of the stem cells or the progenitor cells as a function of the one or more of the biomechanical forces.

6. A method for guiding delivery of a biological agent to biological tissue, the method comprising the steps of:
establishing one or more input parameters by referencing one or more databases;
specifying a device through which the biological agent will be delivered;
determining expected biomechanical forces incurred by the biological agent during delivery through the device;
comparing the expected biomechanical forces to a sensitivity of the biological agent to biomechanical forces;
determining preferred values for one or more controlling parameters from the one or more input parameters in response to the comparing, wherein the one or more controlling parameters are associated with delivering the biological agent through the device; and
providing the preferred values for the one or more controlling parameters to guide the delivery of the biological agent through the device.

7. The method of claim 6, wherein the biological agent comprises one or more of a stem cell, a progenitor cell, or an encapsulated drug.

8. The method of claim 6, wherein the one or more databases comprises interventionalist constraints, clinical constraints, and regulatory agency constraints.

9. The method of claim 6, wherein the one or more databases comprises parameters associated with the biological agent.

10. The method of claim 6, wherein the one or more databases comprises parameters associated with the device through which the biological agent will be delivered.

11. The method of claim 6, wherein determining preferred values for one or more controlling parameters comprises determining a threshold level of shear stress associated with the biological agent.

12. The method of claim 6, wherein determining preferred values for one or more controlling parameters comprises executing a computational optimization routine according to gene expression or phenotype associated with the biological agent.

13. The method of claim 6, wherein determining preferred values for one or more controlling parameters comprises executing a computational optimization routine comprising computing shear stress upon the biological agent.

14. The method of claim 6, wherein determining preferred values for one or more controlling parameters comprises executing a computational optimization routine comprising computing an expected therapeutic efficacy associated with the biological agent.

15. The method of claim 6, further comprising a step of communicating the preferred values for the one or more controlling parameters to the device through which the biological agent is being delivered.

16. The method of claim 6, further comprising a step of communicating the preferred values for the one or more controlling parameters to an interventionalist controlling the device through which the biological agent is being delivered.

17. A computer-based method for guiding delivery of a biological agent to biological tissue, the method comprising the steps of:
receiving a specification of the biological agent, wherein the specification comprises efficacy metrics of the biological agent as a function of shear stress;
evaluating a controlling parameter associated with delivering the biological agent for impact of the controlling parameter on shear stress associated with delivering the biological agent;
determining preferred values for the controlling parameter based on the efficacy metrics evaluated against shear stress associated with delivering the biological agent; and
providing the preferred values for the controlling parameter to guide the delivery of the biological agent.

18. The computer-based method of claim 17, wherein the biological agent comprises one or more of a stem cell, a progenitor cell, or an encapsulated drug.

19. The computer-based method of claim 17, wherein the efficacy metrics comprise changes in phenotype or gene expression.

20. The computer-based method of claim 17, further comprising the step of communicating the preferred values for the controlling parameter to a medical device operable to deliver the biological agent to the biological tissue.
